(19) United States
(12) Patent Application Publication
(10) Pub. No.: US 2026/0349277 A1
(43) Pub. Date: Nov. 5, 2026

(54) GENERATIVE ARTIFICIAL INTELLIGENCE FOR MANUFACTURING INTRAOCULAR LENSES

(71) Applicant: ALCON INC., Fribourg (CH)

(72) Inventors: Robert Dimitri ANGELOPOULOS, San Jose, CA (US); Rachael Claire PETERSON, Fort Worth, TX (US); Bryan STANFILL, Mansfield, TX (US)

(21) Appl. No.: 19/657,134

(22) Filed: Apr. 24, 2026

Related U.S. Application Data
(60) Provisional application No. 63/800,095, filed on May 5, 2025.

(51) Int. Cl. G02C 7/02; G06F 30/27; G06N 3/0475; G16H 10/60; G16H 50/70

(52) U.S. Cl. CPC G02C 7/028; G02C 7/027; G06N 3/0475; G16H 10/60; G16H 50/70; G06F 30/27

(57) ABSTRACT

A computer-implemented method of machine learning based lens optimization. Embodiments include receiving, by a machine learning model, inputs comprising features of a patient. Embodiments include identifying, using a first generative model within the machine learning model, one or more intraocular lens (IOL) designs for the patient, wherein the first generative model has been trained based on IOL design specifications associated with ground truth benchmark data. Embodiments include generating, using a second generative model within the machine learning model, predicted outcome values of the one or more IOL designs for the patient based on the features, wherein the second generative model has been trained based on ground truth patient outcome data for particular IOL designs. Embodiments include forming an IOL according to an IOL design of the one or more IOL designs based on the predicted outcome values.

### INTRODUCTION

The human eye, in its simplest terms, functions to provide vision by transmitting light through a clear outer portion called the cornea, and focusing the resulting image by way of an ocular crystalline lens onto the retina. The quality of the focused image depends on many factors including the size and shape of the eye, and the transparency of the cornea and lens. When age or disease causes the lens to become less transparent, vision deteriorates because of the diminished light which can be transmitted to the retina. This deficiency in the lens of the eye is medically known as a cataract. In addition, the crystalline lens may lose accommodation skills with age, which is called presbyopia. An accepted treatment for those conditions is the surgical removal of the crystalline lens followed by a replacement by an artificial intraocular lens (IOL).

IOL designs may vary in material, power, refractive index, thickness, wavelength, optical zone diameter, and/or other parameters. A given IOL design may produce different outcomes for different patients based upon various factors specific to each patient. While many techniques currently exist for designing and selecting IOLs, continued improvements may offer significant benefits to IOL manufacturing and surgical implantation.

### SUMMARY

In certain embodiments, one general aspect includes a computer-implemented method for machine learning based lens optimization. The computer-implemented method includes: receiving, by a machine learning model, inputs comprising features of a patient; identifying, using a first generative model within the machine learning model, one or more intraocular lens (IOL) designs for the patient, wherein the first generative model has been trained based on IOL design specifications associated with ground truth benchmark data; generating, using a second generative model within the machine learning model, predicted outcome values of the one or more IOL designs for the patient based on the features, wherein the second generative model has been trained based on ground truth patient outcome data for particular IOL designs; and forming an IOL according to an IOL design of the one or more IOL designs based on the predicted outcome values.

In certain embodiments, another general aspect includes a system. The system includes a memory having executable instructions and a processor in communication with the memory. The processor is configured to execute the instructions to perform the computer-implemented method for machine learning based lens optimization described above.

In certain embodiments, another general aspect includes a computer-program product including a non-transitory computer-usable medium having computer-readable program code embodied therein. The computer-readable program code is adapted to be executed to implement the computer-implemented method for machine learning based lens optimization described above.

### BRIEF DESCRIPTION OF THE DRAWINGS

FIG. 1 illustrates an example of computing components related to machine learning based lens optimization, in accordance with certain embodiments of the present disclosure.

FIG. 2 illustrates an example workflow related to machine learning based lens optimization, in accordance with certain embodiments of the present disclosure.

FIG. 3 illustrates an example of a process related to machine learning based lens optimization, in accordance with certain embodiments of the present disclosure.

FIG. 4 illustrates an example of a computing device for machine learning based lens optimization, in accordance with certain embodiments of the present disclosure.

FIG. 5 illustrates an example of a process related to machine learning based lens optimization and formation, in accordance with certain embodiments of the present disclosure.

### DETAILED DESCRIPTION

The present disclosure will now be described in detail with reference to the drawings, which are provided as illustrative examples of the disclosure so as to enable those skilled in the art to practice the disclosure. Notably, the figures and examples below are not meant to limit the scope of the present disclosure to a single embodiment, but other embodiments are possible by way of interchange of some or all of the described or illustrated elements. Moreover, where certain elements of the present disclosure can be partially or fully implemented using known components, only those portions of such known components that are necessary for an understanding of the present disclosure will be described, and detailed descriptions of other portions of such known components will be omitted so as not to obscure the disclosure.

As used herein, the singular form of “a”, “an”, and “the” include plural references unless the context clearly dictates otherwise. As used herein, the statement that two or more parts or components are “coupled” shall mean that the parts are joined or operate together either directly or indirectly (i.e., through one or more intermediate parts or components, so long as a link occurs). As used herein, “directly coupled” means that two elements are directly in contact with each other. As used herein, “fixedly coupled” or “fixed” means that two components are coupled so as to move as one while maintaining a constant orientation relative to each other. As used herein, “operatively coupled” means that two elements are coupled in such a way that the two elements function together. It is to be understood that two elements “operatively coupled” does not require a direct connection or a permanent connection between them. As utilized herein, “substantially” means that any difference is negligible, such that any difference is within an operating tolerance that is known to persons of ordinary skill in the art and provides for the desired performance and outcomes as described in the embodiments described herein. Descriptions of numerical ranges are endpoints inclusive.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality). Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

In the exemplary embodiments described herein, an embodiment showing a singular component should not be considered limiting; rather, the disclosure is intended to encompass other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present disclosure encompasses present and future known equivalents to the known components referred to herein by way of illustration.

Selection of an IOL for a given patient may be based on a variety of factors specific to the patient and the IOL. Certain existing techniques for selecting an IOL for a patient may involve the use of regression models that consider K-values, anterior chamber depth (ACD), axial length, and other physical biometric features that are measured preoperatively. However, these existing approaches generally fall short on predicting which IOL design will have the best outcome in terms of patient visual quality and visual range.

Techniques described herein overcome these challenges through an ensemble of machine learning models that integrates subjective assessments and visual quality in addition to standard pre-operative biometry for machine learning based lens matching. For example, a large eye model (LEM) as described herein may be an ensemble that includes multiple machine learning models trained on different types of data in order to provide improved predictive capability as well as the ability to dynamically generate new IOL designs for particular patients. Models within the LEM may be trained on existing IOL designs, benchmark data, standard biometry, corneal aberration profiles, subjective visual quality assessments, and/or the like. Models within the LEM may incorporate elements of theoretical physics to ensure the physical plausibility of the model outputs, such as applying one or more accuracy checks to model outputs to ensure that inputs to the model and/or outputs from the model are consisent with one or more constraints (e.g., based on one or more principles of theoretical physics). The LEM may enable better matching of IOLs to a given patient and provide a high probability of success for subjective visual outcomes. Furthermore, the LEM may also be used to generate optical design for IOLs that provide better visual outcomes for a larger percentage of the population. These objectives are accomplished by leveraging the particular models within the LEM that are trained on particular factors correlated with patient visual outcomes.

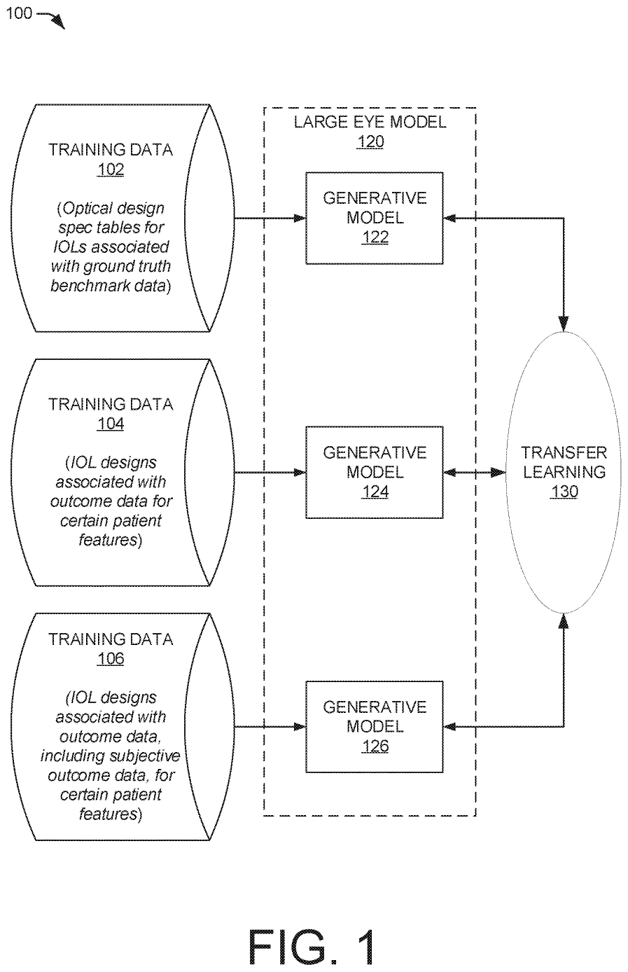

For example, as described in more detail below with respect to FIG. 1, a first generative machine learning model within the large eye model may be trained on IOL designs associated with ground truth benchmark data in order to predict benchmark data for a given IOL design and/or to generate a new IOL design in order to achieve particular target benchmark values. A second generative machine learning model within the large eye model may be trained on IOL designs and features of patients associated with ground truth clinical outcome data (e.g., including objective post-operative measurements) for those patients with those particular IOL designs, such as to predict a clinical outcome for a given patient with a given IOL design. A third generative machine learning model with the large eye model may be trained on IOL designs and features of patients associated with ground truth clinical outcome data (e.g., including objective post-operative measurements as well as subjective outcome data, such as based on feedback from patients, image data, topography data, corneal aberration data, and/or the like) for those patients with those particular IOL designs, such as to predict a clinical outcome for a given patient with a given IOL design with a higher level of resolution than the second generative machine learning model. Transfer learning may be performed among the models in the large eye model to enhance the functioning of each of the individual models and to produce results with a higher level of accuracy. Once trained, the large eye model may be used to optimize selection and/or generation of IOL designs for individual patients with a higher level of accuracy and likelihood of positive clinical outcomes that is possible using prior automated IOL selection techniques.

While existing techniques generally involve the use of a single model, such as a regression model, that is trained on objective patient data such as measured biometric features without the inclusion of any subjective patient outcome data, such techniques may be computationally inefficient and may produce results with suboptimal accuracy. By contrast, techniques described herein utilize a large eye model (LEM) that includes multiple smaller targeted models trained for specific purposes based on data that includes subjective patient outcome information, with transfer learning being used to distill the knowledge from one or more larger models into one or more smaller models within the LEM for improved computational efficiency (e.g. using fewer computing hardware resources such as processing resources) compared to other techniques. Aspects of the present disclosure also improve upon existing techniques for automated IOL selection through a novel combination of machine learning models trained in a particular manner that produces improved accuracy compared to such existing techniques (e.g., compared to a regression model that is not trained based on subject patient outcome data or another type of machine learning model that does not use the particular model combination and/or transfer learning techniques described herein).

Further, the existing technology for IOL selection for a patient typically targets IOL power, but power alone is not the only factor that contributes to visual outcomes. The training of an LEM including individual targeted generative models trained on objective outcomes, corneal aberrations, pupil diameter, subjective outcome data, other pre-operative and post-operative biometry, IOL design specifications, and/or the like enables the LEM to surpass existing automated IOL fitting methods in producing accurate results. The inclusion of subjective assessments in the training data used to train the model(s) enriches the LEM beyond any existing automated IOL calculator. In addition to the ability to predict patient outcomes, the LEM described herein is able to design IOLs, which no IOL calculator can currently do. Thus, the combination of machine learning models in the LEM as described herein, and how such models interact in particular manners described herein, not only improves existing systems and techniques for automated IOL selection, design, and/or manufacturing, but also provides functionality that is currently non-existant in the realm of prediction techniques, especially as it pertains to IOL selection, design, and manufacturing.

The ability to design optics to optimize patient outcomes in an accurate manner that is not possible with prior automated techniques is a clear advantage of techniques described herein. By training the LEM on IOL design parameters and their associated optical performance metrics along with the pre-op and post-op data, aspects of the present disclosure enable the automated optimization of optical designs that enable the maximum portion of the population to achieve highly accurate refractive outcomes, as well as a high probability of patient satisfaction.

FIG. 1 includes an illustration 100 of example computing components related to machine learning based lens optimization, in accordance with certain embodiments of the present disclosure.

In illustration 100, a large eye model (LEM) 120 is an ensemble machine learning model that includes multiple generative models 122, 124, and 126. Generative models 122, 124, and 126 may each be a suitable type of generative machine learning model, such as a generative adversarial network (GAN), large language model (LLM), transformer model, variational autoencoder, autoregressive model, flow model, or another suitable type of machine learning model capable of performing the applicable functionality described herein. Generative models 122, 124, and 126 may be the same type of model or may be different types of models. In one example, generative model 126 has a larger number of parameters than generative model 124 and/or generative model 122. All three models may incorporate theoretical physics to ensure the physical plausibility of the outputs. Examples include but are not limited to, physics-based boundary conditions on input and output variables, and physics-based restrictions on functional relationships between input and output variables. For intstance, a software component (e.g., agent) that executes one or more models in LEM 120 may perform validation of model inputs and/or model outputs using one or more such physics-based boundary conditions on input and/or output variables, and/or one or more such physics-based restrictions on functional relationships between input and output variables. For example, only validated inputs may be provided to the model (and non-validated inputs may be discarded, such as without running the model) and/or only validated outputs may be provided as outputs for further processing (and non-validated outputs may be discarded without being used in further processing).

Each of generative models 122, 124, and 126 may be trained through a supervised learning process based on corresponding training data 102, 104, or 106. In a typical supervised learning process, a set of training inputs is provided to a model, the model generates an output in response to the set of training inputs, the generated output is compared to a label associated with the training inputs, and one or more parameters of the model are adjusted based on the comparing, such as iteratively until one or more conditions are met. For instance, the one or more conditions may relate to an objective function (e.g., a cost function), or may relate to whether the outputs produced by the model based on the training inputs match the labels associated with the training inputs or whether a measure of error between training iterations is not decreasing or not decreasing more than a threshold amount. The conditions may also include whether a training iteration limit has been reached. Parameters adjusted during training may include, for example, hyperparameters, values related to numbers of iterations, weights, functions used by nodes to calculate scores, and the like. In some embodiments, validation and testing are also performed for a machine learning model, such as based on validation data and test data, as is known in the art.

Training data 102, 104, and 106 may be based on historical or prior data, such as including results of past cases in which particular IOLs were used for particular patients, and data related to the outcomes of such cases may be used as labels in the training data.

Training data 102, which may be used to train generative model 122, includes optical design specification tables for IOLs associated with ground truth benchmark data (e.g., the ground truth benchmark data may be the labels in the training data). For example, the IOL design specification tables may include parameters of particular IOL designs such as lens base power, refractive index of lens body, near add power, intermediate add power, spherical aberration correction, cylinder power, material, optical zone diameter, and/or the like. The ground truth benchmark data may include known (e.g., measured or otherwise recorded) benchmark values associated with those particular IOL designs, such as energy distribution, modulation transfer function (MTF), through-focus MTF, point spread function, light utilization, and/or the like. Generative model 122 may be trained through a supervised learning process using training data 102 in order to enable generative model 122 to predict benchmark values for a given IOL design having particular IOL design parameters and/or to generate an IOL design that has certain IOL parameters and that is predicted to produce particular target benchmark values.

Training data 104, which may be used to train generative model 124, includes IOL designs and patient features associated with outcome data for the patients having those patient features and having been treated with IOLs corresponding to those IOL designs (e.g., the outcome data may be the labels in the training data). For example, the IOL designs may include parameters of particular IOL designs such as lens base power, refractive index of lens body, near add power, intermediate add power, spherical aberration correction, cylinder power, material, optical zone diameter, and/or the like, and/or may include benchmark values for particular IOL designs (energy distribution, MTF, through-focus MTF, point spread function, light utilization, and/or the like).

The patient features may include biometric attributes such as pre-operative k-values, pre-operative axial length, pre-operative anterior chamber depth, pre-operative pupil diameter, preoperative manifest refraction spherical equivalent (MRSE), other personal patient attributes, and/or the like. The outcome data may include known (e.g., measured or otherwise recorded) outcome values associated with those particular IOL designs for the patients described by the patient features, such as including post-operative pupil diameter, post-operative manifest refraction spherical equivalent (MRSE), post-operative k-values, post-operative axial length, post-operative anterior chamber depth, and/or the like. Generative model 124 may be trained through a supervised learning process using training data 104 in order to enable generative model 124 to predict outcome values for a given IOL design for a particular patient or for multiple patients having a shared set of characteristics (e.g., based on pre-operative biometric data for the patient(s) and/or other patient attributes). In some embodiments, training data 104 may further include ground truth benchmark data for the IOL designs, and generative model 124 may further be trained to output predicted benchmark values for the IOL designs for particular patients or groups of patients.

Training data 106, which may be used to train generative model 126, includes IOL designs and patient features associated with outcome data, including subjective outcome data (e.g., unlike training data 104), for the patients having those patient features and having been treated with IOLs corresponding to those IOL designs (e.g., the outcome data may be the labels in the training data). For example, the IOL designs may include parameters of particular IOL designs such as lens base power, refractive index of lens body, near add power, intermediate add power, spherical aberration correction, cylinder power, material, optical zone diameter, and/or the like, and/or may include benchmark values for particular IOL designs (energy distribution, MTF, through-focus MTF, point spread function, light utilization, and/or the like). The patient features may include biometric attributes such as pre-operative k-values, pre-operative axial length, preoperative anterior chamber depth, pre-operative pupil diameter, pre-operative manifest refraction spherical equivalent (MRSE), other personal patient attributes, and/or the like.

The patient features may also include subjective data, such as based on patient feedback, including such data as pre-operative uncorrected visual acuity and pre-operative best corrected distance visual acuity. In some aspects, the patient features may also include high-resolution data such as pre-operative image data, pre-operative topography data, pre-operative corneal aberration data, and/or the like. The outcome data may include subjective outcome data, such as based on feedback from patients. For example, subjective outcome data may include post-operative uncorrected visual acuity and post-operative best corrected distance visual acuity. The outcome data may also include high-resolution data such as image data, topography data, corneal aberration data, and/or the like. Furthermore, like training data 104, the outcome data in training data 105 may further include known (e.g., measured or otherwise recorded) outcome values such as post-operative pupil diameter, post-operative manifest refraction spherical equivalent (MRSE), post-operative k-values, post-operative axial length, post-operative anterior chamber depth, and/or the like.

Generative model 126 may be trained through a supervised learning process using training data 106 in order to enable generative model 126 to predict outcome values (e.g., including subjective and/or high-resolution outcome data) for a given IOL design for a particular patient or for multiple patients having a shared set of characteristics (e.g., based on pre-operative biometric data for the patient(s) and/or other patient attributes). In some embodiments, training data 106 may further include ground truth benchmark data for the IOL designs, and generative model 126 may further be trained to output predicted benchmark values for the IOL designs for particular patients or groups of patients.

Furthermore, at transfer learning 130, outputs generated using one or more of generative models 122, 124, and/or 126 may be used to re-train, fine-tune, and/or otherwise further train one or more other models of generative models 122, 124, and/or 126. For example, generative model 124 may be trained based on outputs generated using generative model 126, such as to enrich generative model 124 (which may be a simpler, model that utilizes fewer physical computing resources such as memory and processing resources, such as having fewer parameters than generative model 126) based on knowledge of generative model 126 (which may be relatively more complex and/or may have a larger number of parameters than generative model 124). For example, the knowledge gained through the subjective outcome data and/or higher resolution data used to train generative model 126 may be generalized to a larger number of patients through honing generative model 124 based on transfer learning 130. In another example, the knowledge gained from generative model 124 and/or generative model 126 may be used at transfer learning 130 to further train generative model 122, such as to improve the ability of generative model 122 to predict benchmark values and/or to generate IOL designs.

Transfer learning 130 may generally involve using the output of one model as a label for a new training data instance used to train another model. For example, an output generated by generative model 126 based on inputs related to a particular IOL design and a particular patient may be used as a label for training inputs related to the same IOL design and the same patient in a new training data instance for use in training generative model 124 (e.g., the training inputs for the new training data instance may be a subset of the inputs provided to generative model 126 to generate the output, such as excluding subjective outcome data and/or higher resolution outcome data such as images).

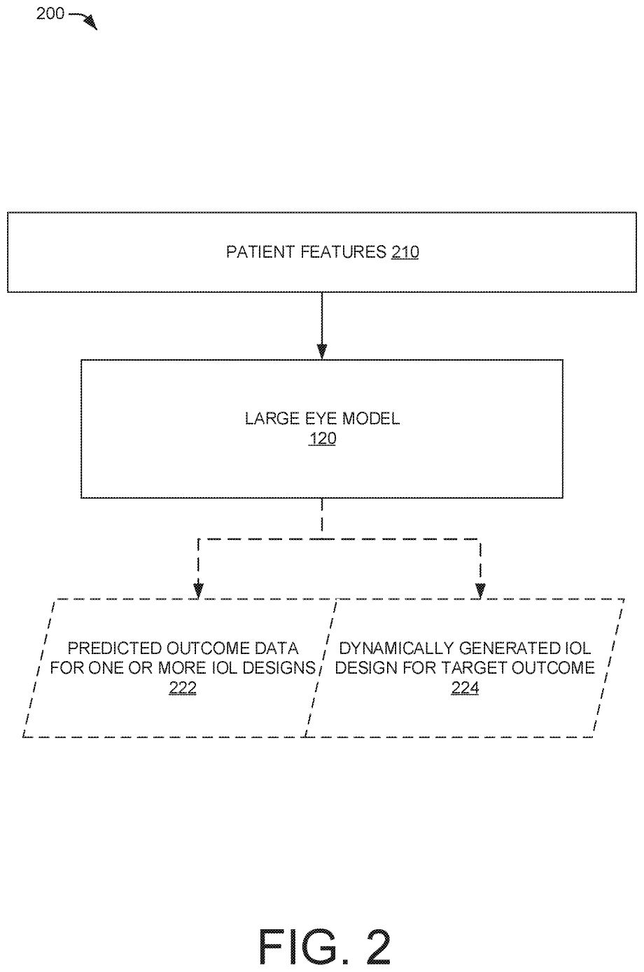

Once trained, LEM 120 may be used to automatically select an IOL design that is likely to produce a target outcome for a particular patient or group of patients and/or to automatically generate an IOL design that is likely to produce a target outcome for a particular patient or group of patients, as described in more detail below with respect to FIG. 2. For example, LEM 120 may be used by an IOL calculator component that is able to perform optimized IOL matching for patients.

LEM 120 may be trained on IOL optic designs such as monofocals, aspheres, torics, extended depth of focus (EDOF), diffractive multifocals, and/or the like. The training process described above enables LEM 120 to design optics that optimize visual outcomes for the largest percentage of the population. There is no prior IOL calculator or other automate component that can design an IOL. LEM 120 may be enabled through training described herein to understand the parameters of ocular geometry and biometry, as well as optical properties like power and aberration profile. In addition, LEM 120 may learn how parameters are distributed across the population and among each other. Further, LEM 120 may be trained through techniques described herein to understand how IOL parameter selection influences the outcome of cataract surgery. Such training may enable LEM 120 to be used as a lens matching and/or generation tool for surgeons or other professionals, such as generating probabilities of achieving target levels of uncorrected visual acuity at specific distances and/or other target outcome values. There is no prior IOL calculator or other automated component that can accomplish such an objective.

As described above, LEM 120 may be generated using a large set of objective data (e.g., which may be included in training data 102 and/or training data 104), a smaller set of objective data with paired subjective data (e.g., which may be included in training data 106), as well as detailed lens design information (e.g., which may be included in training data 102, training data 104, and/or training data 106). The large set of objective data may be used to train a model (e.g., generative model 124) that understands the population level distribution of ocular biometry including but not limited to length, corneal power, anterior chamber size, and their correlations. The smaller set of objective data with associated subjective data may be used to train a model (e.g., generative model 126) that connects objective measurement about pre-operative biometry and post-operative outcomes to subjective measurements about patient satisfaction and vision quality. Overlap between the objective measurements from both datasets will allow the LEM 120 to impute subjective measures to the population level. LEM 120 may then be utilized to predict patient outcomes on a population level for the given IOL designs.

In some cases, the patient biometric information and IOL information is treated as fixed, and the patient outcomes (objective and subjective) are predicted by the LEM 120. The LEM 120 can also be used in the case where the patient biometric and outcome information is fixed but the IOL design is not fixed, allowing the LEM 120 to optimize the lens information given that patient's information. LEM 120 can be used to optimize over a discrete set of lens design options (e.g., those already manufactured by a particular manufacturer or multiple manufacturers), or it can be allowed to optimize over a continuous set of options, some of which may not exist yet (e.g., LEM 120 may generate a design for an IOL that has yet to be formed). Thus, LEM 120 can be used to generate new (non-accommodating) optical designs that maximize patient outcomes. All known IOL optic designs may be used to train LEM 120, these include monofocals, torics, EDOF, and trifocal designs. LEM 120 may be trained on optical power, toricity and spherical aberration properties of each IOL.

With prior techniques, designing an IOL may involve the use of finite element analysis (FEA) in combination with optical design software, which is an iterative process that requires a continuous loop between FEA and optical raytracing software. This iterative process can take hundreds of iterations to find the right optical profile for the dynamic lens. The utilization of LEM 120 as described herein can reduce the amount of time and computing resources (e.g., memory and/or processing resources) required to generate an IOL design significantly, and may optionally be used as an optimization engine for the optic design, such as working between FEA and optical raytracing software.

FIG. 2 illustrates an example workflow 200 related to machine learning based lens optimization, in accordance with certain embodiments of the present disclosure. Workflow 200 includes LEM 120 of FIG. 1, and may represent use of LEM 120 after a training process such as that described above with respect to FIG. 1. It is understood that a variety of machine learning techniques exist for such a training process, and any suitable machine learning algorithm(s) and/or model(s) may be used to train and/or fine-tune LEM 120.

Patient features 210 may be provided as inputs to LEM 120, and LEM 120 may output either predicted outcome data for one or more IOL designs 222 or a dynamically generated IOL design for a target outcome 224. Patient features 210 may include, for example, a pre-operative k-value, a pre-operative axial length, a pre-operative anterior chamber depth, a pre-operative pupil diameter, a pre-operative manifest refraction spherical equivalent (MRSE), one or more other personal patient attribute(s), and/or the like. Patient features 210 may also include subjective data, such as based on patient feedback, including such data as pre-operative uncorrected visual acuity and pre-operative best corrected distance visual acuity. In some aspects, the patient features may also include high-resolution data such as pre-operative image data, pre-operative topography data, pre-operative corneal aberration data, and/or the like. In certain aspects, patient features 210 may also include one or more preferences, such as target values or ranges for one or more outcome values. If the use case involves generating predicted outcome data for one or more IOL designs 222, then one or more IOL designs may also be provided to LEM 120 as inputs. If the use case involves generating a dynamically generated IOL design for a target outcome 224, then LEM 120 may not be provided with an IOL design, but may be provided with one or more preferences, such as target values or ranges for one or more outcome values.

In order to generate predicted outcome data for one or more IOL designs 222 or a dynamically generated IOL design for a target outcome 224, LEM 120 may process the inputs through one or more models (e.g., generative model 122, 124, and/or 126 of FIG. 1) within LEM 120.

In a first example, the use case involves generating predicted outcome data for one or more IOL designs 222. Patient features 210 and one or more IOL designs may be provided as inputs to LEM 120 in the example. The one or more IOL designs may be processed by generative model 122 of FIG. 1, which may output predicted benchmark values for each of the one or more IOL designs. The predicted benchmark values (and, in some aspects, the one or more IOL designs) may be provided along with a subset of patient features 210 (e.g., the objective patient features 210, such as excluding subjective patient features) to generative model 124 of FIG. 1, which may output predicted outcome values (e.g., for objective types of outcome data).

The predicted benchmark values (and, in some aspects, the one or more IOL designs) may be provided along with a subset of patient features 210 (e.g., at least some of the objective patient features 210 along with any subjective patient features 210 and/or high-resolution patient features 210) to generative model 126 of FIG. 1, which may output predicted outcome values (e.g., for certain objective types of outcome data as well as for subjective types of outcome data). Alternatively, generative model 126 of FIG. 1 may be used to train generative model 124 of FIG. 1 (e.g., at transfer learning 130 of FIG. 1), and generative model 126 of FIG. 1 may not itself be used at inference time to generate outputs for a given set of patient features such as patient features 210 (e.g., to save computing resources such as memory and/or processing resources).

The predicted outcome values output by one or more of the generative models in LEM 120 may be provided as predicted outcome data for one or more IOL designs 222. For example, predicted outcome data for one or more IOL designs 222 may indicate, for each IOL design and with respect to the patient represented by patient features 210, a predicted post-operative uncorrected visual acuity, a predicted post-operative best corrected distance visual acuity, predicted post-operative image data, predicted post-operative topography data, predicted post-operative corneal aberration data, predicted post-operative pupil diameter, predicted post-operative manifest refraction spherical equivalent (MRSE), predicted post-operative k-values, predicted post-operative axial length, predicted post-operative anterior chamber depth, and/or the like. In some aspects, predicted outcome data for one or more IOL designs 222 may indicate, for each IOL design and with respect to the patient represented by patient features 210, one or more predicted benchmark values.

In a second example, the use case involves generating a dynamically generated IOL design for a target outcome 224. Patient features 210, including one or more target outcome values (which may include one or more target benchmark values), may be provided as inputs to LEM 120 in the example. One or more of the inputs may be processed through generative model 122 of FIG. 1, which may output one or more dynamically generated IOL designs that are predicted to achieve one or more target benchmark values indicated in the inputs. The one or more generated IOL designs (e.g., along with the target benchmark values) may be provided along with a subset of patient features 210 (e.g., the objective patient features 210, such as excluding subjective patient features) to generative model 124 of FIG. 1, which may output predicted outcome values (e.g., for objective types of outcome data).

The one or more generated IOL designs (e.g., along with the target benchmark values) may be provided along with a subset of patient features 210 (e.g., at least some of the objective patient features 210 along with any subjective patient features 210 and/or high-resolution patient features 210) to generative model 126 of FIG. 1, which may output predicted outcome values (e.g., for certain objective types of outcome data as well as for subjective types of outcome data). Alternatively, generative model 126 of FIG. 1 may be used to train generative model 124 of FIG. 1 (e.g., at transfer learning 130 of FIG. 1), and generative model 126 of FIG. 1 may not itself be used at inference time to generate outputs for a given set of patient features such as patient features 210 (e.g., to save computing resources).

The predicted outcome values output by one or more of the generative models in LEM 120 may be used to select one or more of the one or more dynamically generated IOL designs that produces predicted outcome values most aligned with one or more target values, and each of the one or more selected IOL designs may be output as dynamically generated IOL design for target outcome 224. For example, dynamically generated IOL design for target outcome 224 may indicate parameters of a particular IOL design such as lens base power, refractive index of lens body, near add power, intermediate add power, spherical aberration correction, cylinder power, material, optical zone diameter, and/or the like. Dynamically generated IOL design for target outcome 224 may also indicate benchmark values such as energy distribution, modulation transfer function (MTF), through-focus MTF, point spread function, light utilization, and/or the like.

Thus, LEM 120 may be used by an automated IOL calculator to provide the highest quality visual outcome for a patient, utilizing both objective and subjective metrics. LEM 120 may be also used to design non-accommodating (e.g., static or non-adapting) optics to provide the largest portion of the population the highest probability of satisfaction with their visual outcome post cataract surgery. Furthermore, LEM 120 may be used to design dynamic optics by integrating LEM 120 into a workflow between FEA and optical raytracing software.

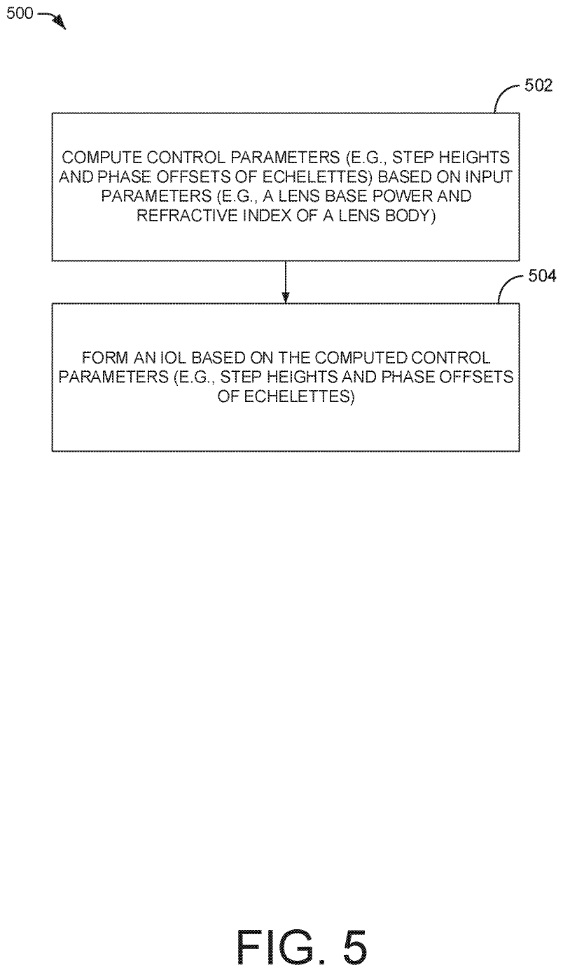

Predicted outcome data for one or more IOL designs 222 or a dynamically generated IOL design for a target outcome 224 may be used to perform one or more actions such as displaying content (e.g., a recommended IOL design or predicted outcome data) via a user interface, selecting an IOL design for implanting in a patient, forming an IOL (e.g., manufacturing an IOL based on a selected or generated IOL design) and/or the like. An example of forming an IOL based on a selected or generated IOL design is described below with respect to FIG. 5. In one example, an IOL may be three-dimensionally printed or otherwise manufactured based on a dynamically generated IOL design for a target outcome 224.

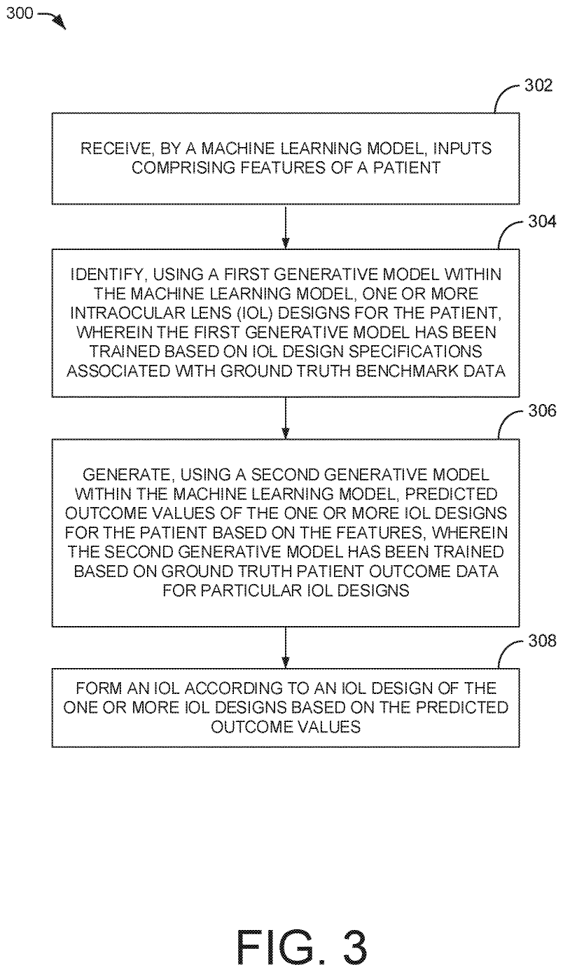

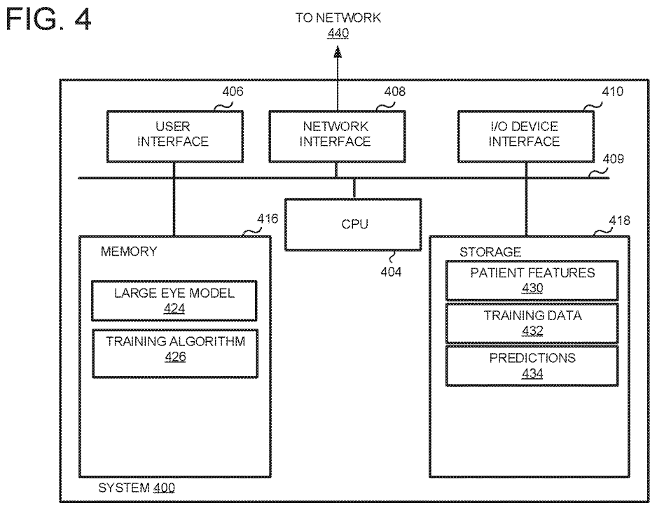

FIG. 3 illustrates an example of a process 300 related to machine learning based lens optimization, in accordance with certain embodiments of the present disclosure. For example, process 300 may be performed by a computing device such as system 400 of FIG. 4 (described below) and/or using LEM 120 of FIGS. 1 and 2. It is noted that any number of systems, in whole or in part, can implement process 300.

Process 300 begins at block 302, with receiving, by a machine learning model, inputs comprising features of a patient.

Process 300 may continue at block 304, with identifying, using a first generative model within the machine learning model, one or more intraocular lens (IOL) designs for the patient, wherein the first generative model has been trained based on IOL design specifications associated with ground truth benchmark data.

Process 300 may continue at block 306, with generating, using a second generative model within the machine learning model, predicted outcome values of the one or more IOL designs for the patient based on the features, wherein the second generative model has been trained based on ground truth patient outcome data for particular IOL designs.

Process 300 may continue at block 308, with forming an IOL according to an IOL design of the one or more IOL designs based on the predicted outcome values.

Some aspects further comprise generating, using a third generative model within the machine learning model, additional predicted outcome values of the one or more IOL designs for the patient, wherein the third generative model has been trained based on additional ground truth patient outcome data for certain IOL designs, and wherein the performing of the action related to the one or more IOL designs is further based on the additional predicted outcome values.

In certain aspects, the additional ground truth patient outcome data is based on feedback provided by one or more patients. In some aspects, the additional ground truth patient outcome data comprises one or more of: image data; topography data; corneal aberration data; or visual acuity data. In some aspects, the second generative model has further been trained based on one or more predictions generated using the third generative model. In some aspects, the first generative model has further been trained based on one or more predictions generated using the second generative model or the third generative model.

In certain aspects, the ground truth patient outcome data or the additional ground truth patient outcome data comprises one or more of: a post-operative pupil diameter; a post-operative manifest refraction spherical equivalent (MRSE); a post-operative k-value; a post-operative axial length; a post-operative anterior chamber depth; a post-operative uncorrected visual acuity; or a post-operative best corrected distance visual acuity.

In some aspects, the predicted outcome values of the one or more IOL designs for the patient or the additional predicted outcome values of the one or more IOL designs for the patient comprise one or more of: a predicted post-operative pupil diameter; a predicted post-operative manifest refraction spherical equivalent (MRSE); a predicted post-operative k-value; a predicted post-operative axial length; a predicted post-operative anterior chamber depth; a predicted post-operative uncorrected visual acuity; or a predicted post-operative best corrected distance visual acuity.

In certain aspects, the identifying of the one or more IOL designs for the patient comprises dynamically generating, by the first generative model, at least one IOL design of the one or more IOL designs.

In some aspects, one or more of the first generative model or the second generative model comprises a generative adversarial network.

In certain aspects, the features of the patient comprise one or more biometric attributes of the patient. In some aspects, the one or more biometric attributes of the patient comprises one or more of: a pre-operative k-value; a pre-operative axial length; or a pre-operative anterior chamber depth.

In some aspects, the one or more IOL designs comprise one or more of: a spherical IOL model; an aspheric IOL model; a toric IOL model; or a diffractive multifocal IOL model.

In certain aspects, the IOL design specifications comprise one or more of: lens base power data; cylinder power data; add power data; material data; refractive index data; spherical aberration correction data; or optical zone diameter data.

In some aspects, the ground truth benchmark data comprises one or more of: spherical aberration data; energy distribution data; modulation transfer function (MTF) data; through-focus MTF data; point spread function data; or light utilization data.

In certain aspects, in addition to or instead of forming the IOL, the process may further include one or more of: displaying an IOL design recommendation via a user interface based on the predicted outcome values; or selecting the IOL design of the one or more IOL designs based on the predicted outcome values.

Some aspects further comprise validating an input to or an output from the first generative model or the second generative model according to one or more physics-based boundary conditions or one or more physics-based restrictions on a functional relationship between the input and the output.

FIG. 4 illustrates an example of a system 400 for machine learning based lens optimization, in accordance with certain embodiments of the present disclosure. For example, system 400 may be configured to perform process 300 of FIG. 3 and/or other aspects of the present disclosure, such as discussed above with respect to FIGS. 1-2.

As shown, system 400 includes, without limitation, central processing unit (CPU) 404, user interface 406, network interface 408, memory 416, storage 418, interconnect 409, and at least one I/O device interface 410 which may allow for the connection of various I/O devices (e.g., keyboards, displays, mouse devices, pen input, etc.) to system 400. While one or more operations are described herein as being performed by particular components of system 400, those operations may, in some embodiments, be performed by other components of system 400 and/or component(s) of other system(s). As an example, while one or more operations are described herein as being performed by CPU 405, memory 416, and/or storage 418 those operations may, in other embodiments, be performed by other components of system 400 or of a different system.

CPU 404 may be representative of one or more processing devices and/or cores. In some embodiments, CPU 404 may retrieve and execute programming instructions stored in memory 416. Similarly, CPU 404 may retrieve and store application data residing in memory 416. Interconnect 409 transmits programming instructions and application data, among CPU 404, I/O device interface 410, user interface 406, memory 416, storage 418, network interface 408, etc. In some embodiments, CPU 404 may correspond to a single CPU, multiple CPUs, or a single CPU having multiple processing cores. Additionally, in some embodiments, memory 416 represents volatile memory, such as random-access memory. In some embodiments, storage 418 may be non-volatile memory, such as a disk drive, solid state drive, or a collection of storage devices distributed across multiple storage systems.

System 400 can include a network interface 408 for connection with a data communications network (e.g., network 470), such as to communicate with other devices. The data communications network can be, or can include, one or more of a private network, a public network, a local or wide area network, the Internet, combinations of the same, and/or the like. The data communications network can include, for example, interfaces (e.g., application programming interfaces) for enabling interaction and communication between and among the components and systems of a computing environment (e.g., of FIG. 1) and/or other components and systems.

The memory 516 can include a LEM 424, which generally represents LEM 120 of FIGS. 1 and 2. The memory 516 can further include a training algorithm 426, which generally represents one or more training algorithms for LEM 424, such as those described above with respect to FIG. 1.

The storage 418 can include patient features 430, which may include patient features 210 of FIG. 1. The storage 418 can further include training data 432, which may include training data 102, 104, and/or 106 of FIG. 1. The storage 418 can further include predictions 434, which may include predicted outcome data for one or more IOL designs 222 and/or dynamically generated IOL design for a target outcome 224 of FIG. 2.

It is noted that system 400 is included as an example, and techniques described herein may be implemented via fewer or more components, either on the same or different devices, and devices may include physical and/or virtual devices.

FIG. 5 illustrates an example of a process 500 related to machine learning based lens optimization and formation, in accordance with certain embodiments of the present disclosure. For example, process 500 may be performed by a computing device such as system 400 of FIG. 4 (described above), using LEM 120 of FIGS. 1 and 2, and/or by one or more other components or systems. It is noted that any number of systems, in whole or in part, can implement process 500. In some embodiments, step 502 of operations 500 is performed by one system (e.g., system 400 of FIG. 4) while step 504 is performed by a lens manufacturing system. In other embodiments, both steps 502 and 504 are performed by a lens manufacturing system.

At step 502, control parameters that define the structure of an ocular lens (e.g., IOL) are computed based on a set of input parameters. Example control parameters include step heights, phase offsets of echelettes, a number of echelettes, central refractive element radius (R_{c}), peripheral refractive element radius (Rₒ), and/or diffractive grating structure radius, of an ocular lens (e.g., an IOL). Example input parameters may include lens base power, refractive index of lens body, near add power, intermediate add power, spherical aberration correction, cylinder power, material, optical zone diameter, and/or the like (e.g., the input parameters may be specified in an IOL design, such as one selected or generated using techniques described above with respect to FIGS. 1-4). The computations performed at step 502 are based on one or more of the embodiments described herein.

At step 504, an ocular lens (e.g., an IOL) is formed based on the input parameters as well as the computed control parameters (e.g., number of echelettes, central refractive element radius (R_{c}), peripheral refractive element radius (Rₒ), diffractive grating structure radius, step heights and phase offsets of diffractive grating structure echelettes) using appropriate methods, systems, and devices typically used for manufacturing lenses, as known to one of ordinary skill in the art.

As used herein, a phrase referring to “at least one of” a list of items refers to any combination of those items, including single members. As an example, “at least one of: a, b, or c” or “at least one of: a, b, and c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims.

Within a claim, reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.” The word “exemplary” is used herein to mean “serving as an example, instance, or illustration.” Any aspect described herein as “exemplary” is not necessarily to be construed as preferred or advantageous over other aspects.

What is claimed is:

1. A system for machine learning based lens optimization, the system comprising:
one or more processors; and
a memory storing instructions that, when executed by the one or more processors, cause the system to:
receive, by a machine learning model, inputs comprising features of a patient;
identify, using a first generative model within the machine learning model, one or more intraocular lens (IOL) designs for the patient, wherein the first generative model has been trained based on IOL design specifications associated with ground truth benchmark data;
generate, using a second generative model within the machine learning model, predicted outcome values of the one or more IOL designs for the patient based on the features, wherein the second generative model has been trained based on ground truth patient outcome data for particular IOL designs; and
forming an IOL according to an IOL design of the one or more IOL designs based on the predicted outcome values.

2. The system of claim 1, wherein the instructions, when executed by the one or more processors, further cause the system to generate, using a third generative model within the machine learning model, additional predicted outcome values of the one or more IOL designs for the patient, wherein the third generative model has been trained based on additional ground truth patient outcome data for certain IOL designs, and wherein the performing of the action related to the one or more IOL designs is further based on the additional predicted outcome values.

3. The system of claim 2, wherein the additional ground truth patient outcome data is based on feedback provided by one or more patients.

4. The system of claim 2, wherein the additional ground truth patient outcome data comprises one or more of:
image data;
topography data;
corneal abberration data; or
visual acuity data.

5. The system of claim 2, wherein the second generative model has further been trained based on one or more predictions generated using the third generative model.

6. The system of claim 2, wherein the first generative model has further been trained based on one or more predictions generated using the second generative model or the third generative model.

7. The system of claim 2, wherein the ground truth patient outcome data or the additional ground truth patient outcome data comprises one or more of:
a post-operative pupil diameter;
a post-operative manifest refraction spherical equivalent (MRSE);
a post-operative k-value;
a post-operative axial length;
a post-operative anterior chamber depth;
a post-operative uncorrected visual acuity; or
a post-operative best corrected distance visual acuity.

8. The system of claim 2, wherein the predicted outcome values of the one or more IOL designs for the patient or the additional predicted outcome values of the one or more IOL designs for the patient comprise one or more of:
a predicted post-operative pupil diameter;
a predicted post-operative manifest refraction spherical equivalent (MRSE);
a predicted post-operative k-value;
a predicted post-operative axial length;
a predicted post-operative anterior chamber depth;
a predicted post-operative uncorrected visual acuity; or
a predicted post-operative best corrected distance visual acuity.

9. The system of claim 1, wherein the identifying of the one or more IOL designs for the patient comprises dynamically generating, by the first generative model, at least one IOL design of the one or more IOL designs.

10. The system of claim 1, wherein one or more of the first generative model or the second generative model comprises a generative adversarial network.

11. The system of claim 1, wherein the features of the patient comprise one or more biometric atttributes of the patient.

12. The system of claim 11, wherein the one or more biometric attributes of the patient comprises one or more of:
a pre-operative k-value;
a pre-operative axial length; or
a pre-operative anterior chamber depth.

13. The system of claim 1, wherein the one or more IOL designs comprise one or more of:
a spherical IOL model;
an aspheric IOL model;
a toric IOL model; or
a diffractive multifocal IOL model.

14. The system of claim 1, wherein the IOL design specifications comprise one or more of:
lens base power data;
cylinder power data;
add power data;
material data;
refractive index data;
spherical aberration correction data; or
optical zone diameter data.

15. The system of claim 1, wherein the ground truth benchmark data comprises one or more of:
spherical aberration data;
energy distribution data;
modulation transfer function (MTF) data;
through-focus MTF data;
point spread function data; or
light utilization data.

16. The system of claim 1, wherein the instructions, when executed by the one or more processors, further cause the system to:
display an IOL design recommendation via a user interface based on the predicted outcome values; or
select the IOL design of the one or more IOL designs based on the predicted outcome values.

17. The system of claim 1, wherein the instructions, when executed by the one or more processors, further cause the system to validate an input to or an output from the first generative model or the second generative model according to one or more physics-based boundary conditions or one or more physics-based restrictions on a functional relationship between the input and the output.

18. A computer-implemented method of machine learning based lens optimization, the computer-implemented method comprising:
receiving, by a machine learning model, inputs comprising features of a patient;
identifying, using a first generative model within the machine learning model, one or more intraocular lens (IOL) designs for the patient, wherein the first generative model has been trained based on IOL design specifications associated with ground truth benchmark data;
generating, using a second generative model within the machine learning model, predicted outcome values of the one or more IOL designs for the patient based on the features, wherein the second generative model has been trained based on ground truth patient outcome data for particular IOL designs; and
forming an IOL according to an IOL design of the one or more IOL designs based on the predicted outcome values.

19. The computer-implemented method of claim 18, further comprising generating, using a third generative model within the machine learning model, additional predicted outcome values of the one or more IOL designs for the patient, wherein the third generative model has been trained based on additional ground truth patient outcome data for certain IOL designs, and wherein the performing of the action related to the one or more IOL designs is further based on the additional predicted outcome values.

20. A non-transitory computer-readable medium comprising instructions that, when executed via one or more processors of a computing system, cause the computing system to:
receive, by a machine learning model, inputs comprising features of a patient;
identify, using a first generative model within the machine learning model, one or more intraocular lens (IOL) designs for the patient, wherein the first generative model has been trained based on IOL design specifications associated with ground truth benchmark data;
generate using a second generative model within the machine learning model, predicted outcome values of the one or more IOL designs for the patient based on the features, wherein the second generative model has been trained based on ground truth patient outcome data for particular IOL designs; and
form an IOL according to an IOL design of the one or more IOL designs based on the predicted outcome values.
